(19) United States
(12) Patent Application Publication
(10) Pub. No.: US 2025/0018341 A1
(43) Pub. Date: Jan. 16, 2025

(54) METHOD AND SYSTEM FOR CAPTURING AND UTILIZING CARBON DIOXIDE

(71) Applicant: XECA TURBO TECHNOLOGIES (BEIJING) CO., LTD., Beijing (CN)

(72) Inventor: Xijia LV, Beijing (CN)

(73) Assignee: XECA TURBO TECHNOLOGIES (BEIJING) CO., LTD., Beijing (CN)

(21) Appl. No.: 18/902,181

(22) Filed: Sep. 30, 2024

Related U.S. Application Data
(63) Continuation of application No. PCT/CN2022/096936, filed on Jun. 2, 2022.

(51) Int. Cl. B01D 53/96; B01D 53/14; B01D 53/18; B01D 53/62; B01D 53/73; B01D 53/78; C07C 29/151; C25B 1/04; C25B 15/08

(52) U.S. Cl. CPC B01D 53/965; B01D 53/1425; B01D 53/1475; B01D 53/1493; B01D 53/18; B01D 53/62; B01D 53/73; B01D 53/78; C07C 29/1518; B01D 2251/304; B01D 2251/306; B01D 2251/604; B01D 2257/504; B01D 2258/06; C25B 1/04; C25B 15/08

(57) ABSTRACT

Disclosed are a method and a system for capturing and utilizing carbon dioxide. The method for capturing and utilizing the carbon dioxide includes: performing a capture process on carbon dioxide in a target component by using an alkaline solution, to obtain an aqueous solution containing a carbonate; performing an electrolytic regeneration process on the aqueous solution containing the carbonate, to obtain an aqueous solution of a hydroxide, carbon dioxide produced by electrolysis, oxygen and hydrogen; and performing a catalytic reaction of the carbon dioxide produced by the electrolysis and the hydrogen, to obtain a hydrocarbon.

### CROSS-REFERENCE TO RELATED APPLICATIONS

The present application is a continuation of International Application No. PCT/CN2022/096936, filed on Jun. 2, 2022, which claims priority to Chinese Patent Application No. 202110360301.5, filed on Apr. 2, 2021. The disclosures of the aforementioned applications are hereby incorporated by reference in their entireties.

### TECHNICAL FIELD

The present disclosure relates to the field of carbon dioxide capture and application technologies, and in particular, to a method for capturing and utilizing carbon dioxide and a system for capturing and utilizing carbon dioxide.

### BACKGROUND

Conventional carbon capture technologies are classified into two paths: capturing carbon from a tail gas from burning and capturing carbon from air.

Methods for capturing carbon dioxide from the tail gas from burning are mainly classified into a liquid amine adsorption method and a solid membrane adsorption method. The two methods for capturing carbon dioxide from the tail gas from burning can both reach a pilot plant test level on domestic and overseas, but they cannot solve following problems: since a concentration of the carbon dioxide in the air is merely 400 ppm, an conventional method for capturing carbon from fumes cannot realize capturing the carbon dioxide from the air; a large amount of steam is needed for regeneration of an adsorbent of liquid amine, so that a large amount of heat with low-temperature is required, but generation of the heat may increase emission of the carbon dioxide, and a manufacture cost and an operation cost of a system are increased; and the captured carbon dioxide cannot be utilized on site, and it is necessary to transport the carbon dioxide and find an utilization or storage solution of the carbon dioxide.

The technologies for capturing the carbon dioxide from the air are mainly classified into routes of a liquid alkaline solution adsorption and a solid amine membrane adsorption. The technologies of the two routes are in a pilot plant test stage at present, but the existing technologies for capturing the carbon dioxide from the air cannot solve following problems.

Firstly, since the conventional technologies merely can capture the carbon dioxide from the air, there is no industrial by-product for generating profit, so that a capture cost of the existing technologies is too high.

Secondly, the conventional technologies cannot solve the problems of transportation and utilization of the carbon dioxide, and the captured carbon dioxide needs to be provided with other technologies to solve the problem of the utilization of the carbon dioxide.

Thirdly, in the solid membrane adsorption technology, regeneration of an amine adsorbent requires a large amount of steam with low-temperature during regeneration, so that energy consumption is large. If a heat source of the steam is from the burning of fossil energy, the emission of the carbon dioxide will be increased.

Fourthly, in the conventional alkaline solution adsorption technology, the regeneration of a gas adsorbent needs to be realized through the following two chemical loops: K₂CO₃+Ca(OH)₂=CaCO₃+2KOH, CaCO₃=CaO+CO₂, CaO+H₂O═Ca(OH)₂. Defects of this method are as follows: first, a design of a system is complex, a cost is high, and a control system is difficult to be implemented. Second, the chemical loops of the carbon dioxide and calcium carbonate are realized by combustion at 900° C., so that energy consumption and carbon emission is greatly increased, and the calcium oxide adsorbent is easy to deactivate, resulting in requiring a large amount of supplement of the calcium carbonate.

Lastly, in the alkaline solution adsorption technologies reported in existing documents, an alkaline solution is regenerated by the reaction of chlorine with an aqueous solution of sodium carbonate, and chlorine and sodium hydroxide are obtained by electrolyzing brine (in a chlor-alkali industry). On one hand, this route has problems of having a high investment cost, a complex system and difficulty in realizing accurate control. On the other hand, the chlorine has characteristics of toxicity, corrosion and difficulty in transportation, which will lead to a too high investment cost of safety protection of the system, thus being not conducive to commercialization of the technology.

In view of the above problems, it is necessary to develop a method and system for capturing and utilizing the carbon dioxide which are capable of solving the transportation and utilization of the carbon dioxide and achieving a higher economic value.

### SUMMARY

A main purpose of the present disclosure is to provide a method and system for capturing and utilizing carbon dioxide, to solve problems that conventional methods and apparatuses for capturing carbon dioxide cannot perform transportation and utilization of the carbon dioxide and the system has a high operating cost.

In an industry of green hydrogen, in a process that hydrogen is produced by electrolysis of water with a renewable energy, storage and transportation costs of the hydrogen are very high. The used of the carbon dioxide, as an industrial raw material for realizing the storage of the hydrogen through a liquid organic, has been widely concerned as a technical route which may reduce the storage cost of the hydrogen in the future. However, the carbon dioxide cannot be generated in the process that the hydrogen is produced by the electrolysis of the water, and it needs to be transported to a hydrogen production site, so that the transportation cost is greatly increased. Therefore, the carbon dioxide is capable of being generated simultaneously in a process of producing hydrogen is a key to realize the storage of the hydrogen through the liquid organic at a low cost.

To achieve the foregoing purpose, an aspect of the present disclosure provides a method for capturing and utilizing carbon dioxide, including: performing, by using an alkaline solution, a capture process on carbon dioxide in a target component, to obtain an aqueous solution containing a carbonate; performing, on the aqueous solution containing the carbonate, an electrolytic regeneration process, to obtain an aqueous solution of a hydroxide, carbon dioxide produced by electrolysis, oxygen and hydrogen, and controlling a concentration of the aqueous solution containing the carbonate and an electrolytic voltage in the electrolytic regeneration process to adjust an output ratio of the carbon dioxide produced by the electrolysis and the hydrogen at the same time; and optionally, performing, a catalytic reaction of the carbon dioxide produced by the electrolysis and the hydrogen, to obtain a hydrocarbon, where the hydrocarbon is used as an industrial by-product.

Further, the electrolytic regeneration process includes: performing, on the aqueous solution containing the carbonate, the electrolytic regeneration process, to obtain a mixed gas of the carbon dioxide produced by the electrolysis and the oxygen, the hydrogen and the aqueous solution of the hydroxide; and performing, on the mixed gas, a separation process, to separate the carbon dioxide produced by the electrolysis and the oxygen.

Further, a method of the separation process is selected from one or more of a cryogenic liquefaction method, a catalytic oxidation method, a membrane separation method and a method performed by an adsorption apparatus.

Further, the electrolytic regeneration process is a staged electrolysis process.

Further, in the electrolytic regeneration process, a voltage of an electrolytic cell ranges from 1.5V to 4V, preferably, ranges from 2V to 3V, a current density ranges from 1000 A/m² to 10000 A/m², a hydrogen ion concentration (pH) of the aqueous solution containing the carbonate ranges from 7 to 14, and a concentration of the carbonate in the aqueous solution containing the carbonate ranges from 1 mol/L to 10 mol/L.

Further, the current density ranges from 1500 A/m² to 10000 A/m²; the pH of the aqueous solution containing the carbonate ranges from 7 to 12, and preferably ranges from 7 to 10 or ranges from 8 to 12; in the aqueous solution containing the carbonate, the concentration of the carbonate ranges from 1 mol/L to 6.2 mol/L, and preferably ranges from 1 mol/L to 5 mol/L.

Further, the current density ranges from 2000 A/m² to 6000 A/m², and preferably ranges from 2000 A/m² to 4000 A/m².

Further, before performing the electrolytic regeneration process, the method for capturing and utilizing the carbon dioxide further includes: performing, on the aqueous solution containing the carbonate, an impurity removal process.

Further, after the impurity removal process, a content of alkaline earth metal ions in the aqueous solution containing the carbonate is less than or equal to 10 ppm.

Further, after the impurity removal process, a content of impurity ions in the aqueous solution containing the carbonate is less than or equal to 10 ppm, and the impurity ions include alkaline earth metal ions, Al³⁺ and/or Si⁴⁺.

Further, the alkaline earth metal ions include Ca²⁺ and/or Mg²⁺.

Further, a method of the impurity removal process is selected from a method of filtration, a method of precipitation or a method of adsorption; and preferably, the precipitation is chemical precipitation.

Further, the electrolytic regeneration process is performed under a pressure ranges from 1 atm to 40 bar, and preferably ranges from 2 bar 40 bar.

Further, between the impurity removal process and the electrolytic regeneration process, the method for capturing and utilizing the carbon dioxide further includes: performing, on a concentration of the aqueous solution containing the carbonate, an adjustment process, where a method of the adjustment process includes diluting with water or concentrating by heating.

Further, the method for capturing and utilizing carbon dioxide further includes: performing, on a portion of the aqueous solution containing the carbonate, an extraction process, to obtain the carbonate, where the carbonate is used as an industrial by-product.

Further, a method for the extraction process is a recrystallization method or a crystallization method.

Further, the alkaline solution is an aqueous solution of an alkali metal hydroxide.

Further, the alkaline solution is an aqueous solution of sodium hydroxide and/or an aqueous solution of potassium hydroxide.

Further, a pH of the alkaline solution ranges from 7 to 14, and preferably is greater than 7 and less than or equal to 10.

Further, products of the electrolytic regeneration process further include a bicarbonate.

Further, the electrolytic regeneration process is performed in an electrolytic cell, and the method for capturing and utilizing the carbon dioxide further includes: preheating, by using a portion of the aqueous solution of the hydroxide leaving the electrolytic cell, the aqueous solution containing the carbonate entering the electrolytic cell.

Further, the method for capturing and utilizing the carbon dioxide further includes: taking, as the alkaline solution, the portion of the aqueous solution of the hydroxide leaving the electrolytic cell.

Further, the electrolytic regeneration process is performed in an electrolytic cell, and the method for capturing and utilizing the carbon dioxide further includes: preheating, by using a heat released by the catalytic reaction, the aqueous solution containing the carbonate entering the electrolytic cell.

Further, the method for capturing and utilizing the carbon dioxide further includes: preheating the aqueous solution containing the carbonate to a temperature ranging from 60° C. to 90° C.

Further, the hydrocarbon is selected from one or more of methane, methanol, gasoline and aviation fuel.

Further, when the hydrocarbon is methanol, a temperature in the catalytic reaction ranges from 200° C. to 400° C., a pressure ranges from 10 bar to 50 bar, and a molar ratio of the carbon dioxide produced by the electrolysis to the hydrogen ranges from 1:1 to 1:5.

Further, the target component is selected from air and/or a tail gas from burning.

Further, when the target component is the air, before the capture process is performed, the method for capturing and utilizing the carbon dioxide further includes: performing, on the target component, a concentration process, to obtain a concentrated gas to increase a concentration of the carbon dioxide, and then performing the capture process on the concentrated gas.

Further, the concentration process includes: adsorbing, by using an adsorbent, the carbon dioxide in the target component, and then desorbing to obtain the concentrated gas, where a concentration of the carbon dioxide in the concentrated gas ranges from 0.4% to 5%.

Further, between the concentration process and the capture process, the method for capturing and utilizing the carbon dioxide further includes: performing, on the concentrated gas, a compression process.

Further, a pressure of the compression process ranges from 5 bar to 500 bar.

Further, when the target component is the tail gas from burning, before the capture process is performed, the method for capturing and utilizing the carbon dioxide further includes: performing, a denitrification treatment and desulfurization treatment, and/or a dust removal treatment on the tail gas from burning.

Further, the catalytic reaction is a thermocatalytic reaction, a photocatalytic reaction or a biocatalytic reaction; and the hydrogen used in the catalytic reaction is generated by the electrolytic regeneration process, or is partially generated by the electrolytic regeneration process and a remaining portion is transported from outside, or is completely transported from outside.

Another aspect of the present disclosure provides a system for capturing and utilizing carbon dioxide, including: a carbon dioxide capture apparatus, an electrolytic regeneration unit, a voltage regulating apparatus and optionally a catalytic apparatus. The carbon dioxide capture apparatus is provided with an inlet for an alkaline solution, an inlet for a target component and a discharge port for an aqueous solution containing a carbonate. The electrolytic regeneration unit is provided with a first inlet for the aqueous solution containing the carbonate, an outlet for carbon dioxide produced by electrolysis, an outlet for oxygen, an outlet for hydrogen and a discharge port for an aqueous solution of hydroxide, and the first inlet for the aqueous solution containing the carbonate is in communication with the discharge port for the aqueous solution containing the carbonate through a transport pipeline of the aqueous solution containing the carbonate. The voltage regulating apparatus is configured to regulate a voltage in an electrolytic regeneration process to adjust an output ratio of the carbon dioxide produced by electrolysis and the hydrogen. The catalytic apparatus is provided with a catalytic inlet and an outlet for the hydrocarbon, and the catalytic inlet is respectively in communication with the outlet for the carbon dioxide produced by the electrolysis and the outlet for the hydrogen.

Further, when the voltage regulating apparatus is capable of making the electrolytic regeneration unit perform a staged electrolysis process, the electrolytic regeneration unit is an electrolytic regeneration apparatus, and the electrolytic regeneration apparatus is provided with the first inlet for the aqueous solution containing the carbonate, the outlet for the hydrogen, the outlet for the carbon dioxide produced by the electrolysis, the outlet for the oxygen and the discharge port for the aqueous solution of the hydroxide; or when the electrolytic regeneration unit does not perform a staged electrolysis process, the electrolytic regeneration unit includes an electrolytic regeneration apparatus and a separation apparatus. The electrolytic regeneration apparatus is provided with the first inlet for the aqueous solution containing the carbonate, the outlet for the hydrogen, a discharge port for anode gases and the discharge port for the aqueous solution of the hydroxide, where the anode gases include the carbon dioxide produced by electrolysis and the oxygen. The separation apparatus is provided with an inlet for a gas to be separated, an outlet for the carbon dioxide produced by the electrolysis and an outlet the oxygen, and the inlet for the gas to be separated is in communication with the discharge port for the anode gas.

Further, the separation apparatus is selected from one or more of a cryogenic apparatus, a catalytic oxidation apparatus, an adsorption apparatus and a membrane separation apparatus.

Further, the system for capturing and utilizing the carbon dioxide further includes an impurity removal apparatus. The impurity removal apparatus is disposed on the transport pipeline of the aqueous solution containing the carbonate.

Further, the impurity removal apparatus is selected from a filtration apparatus, a precipitation apparatus or an adsorption apparatus.

Further, the system for capturing and utilizing the carbon dioxide further includes a first heat exchange apparatus. The first heat exchange apparatus is disposed on the transport pipeline of the aqueous solution containing the carbonate.

Further, a hot medium inlet for the first heat exchange apparatus is connected to the discharge port for the aqueous solution of the hydroxide to make the aqueous solution of the hydroxide discharged from the discharge port for the aqueous solution of the hydroxide exchange heat with the aqueous solution containing the carbonate in the transport pipeline of the aqueous solution containing the carbonate.

Further, the system for capturing and utilizing the carbon dioxide includes the catalytic apparatus and a second heat exchange apparatus. The second heat exchange apparatus is disposed on the transport pipeline of the aqueous solution containing the carbonate.

Further, a hot medium inlet for the second heat exchange apparatus is connected to the outlet for the hydrocarbon to make the hydrocarbon discharged from the outlet for the hydrocarbon exchange heat with the aqueous solution containing the carbonate in the transport pipeline of the aqueous solution containing the carbonate.

Further, the system for capturing and utilizing the carbon dioxide further includes a concentration adjusting apparatus of the aqueous solution containing the carbonate disposed on the transport pipeline of the aqueous solution containing the carbonate. The concentration adjusting apparatus of the aqueous solution containing the carbonate is configured to adjusting a concentration and a pH of the aqueous solution containing the carbonate.

Further, the concentration adjusting apparatus of the aqueous solution containing the carbonate is a dilution apparatus or a concentration apparatus.

Further, the system for capturing and utilizing the carbon dioxide further includes a carbonate extraction apparatus. The carbonate extraction apparatus is provided with a second inlet for the aqueous solution containing the carbonate, and the second inlet for the aqueous solution containing the carbonate is in communication with the discharge port for the aqueous solution containing the carbonate.

Further, the extraction apparatus is a crystallization apparatus or a recrystallization apparatus.

Further, the discharge port for the aqueous solution of the hydroxide is in communication with the inlet for the alkaline solution.

Further, when the target component is air, the system for capturing and utilizing the carbon dioxide further includes a concentration unit. The concentration unit is provided with an inlet for a gas to be concentrated and an outlet for a concentrated gas, the outlet for the concentrated gas is in communication with the inlet for the target component through a transport pipeline of the concentrated gas, and the concentration unit is configured to improve a content of the carbon dioxide in the target component.

Further, the concentration unit includes a carbon dioxide adsorption apparatus and a desorption apparatus. The carbon dioxide adsorption apparatus is provided with the inlet for the gas to be concentrated, and is configured to adsorb the carbon dioxide in the target component. The desorption apparatus is disposed downstream of the carbon dioxide adsorption apparatus and is provided with an outlet for the concentrated gas, which is configured to desorb the carbon dioxide being adsorbed in the carbon dioxide adsorption apparatus.

Further, the system for capturing and utilizing the carbon dioxide further includes a first compression apparatus. The first compression apparatus is disposed on the transport pipeline of the concentrated gas.

Further, when the target component is a tail gas from burning, the system for capturing and utilizing the carbon dioxide further includes: a dust removal apparatus, a desulfurization apparatus, a denitrification apparatus and a transport pipeline of the target component in communication with the inlet for the target component. The dust removal apparatus, the desulfurization apparatus and the denitrification apparatus are disposed on the transport pipeline of the target component.

Further, the system for capturing and utilizing the carbon dioxide further includes a collection apparatus. The collection apparatus is provided with a collection port, and the collection port is in communication with the oxygen outlet for collecting oxygen.

Further, the system for capturing and utilizing the carbon dioxide further includes a carbon dioxide compression apparatus and a hydrogen compression apparatus. The carbon dioxide compression apparatus is provided with an inlet for the carbon dioxide produced by the electrolysis and an outlet for compressed carbon dioxide, the inlet for the carbon dioxide produced by the electrolysis is in communication with the outlet for the carbon dioxide produced by the electrolysis of the electrolytic regeneration unit, and the outlet for the compressed carbon dioxide is in communication with the catalytic inlet. The hydrogen compression apparatus is provided with an inlet for hydrogen and an outlet for compressed hydrogen, the inlet for the hydrogen is in communication with the outlet for the hydrogen of the electrolytic regeneration unit, and the outlet for the compressed hydrogen is in communication with the catalytic inlet.

According to the method for capturing and utilizing the carbon dioxide provided by the present disclosure, not only emission reduction of the carbon dioxide is capable of being realized, but also optionally the problems of the transportation and utilization of the carbon dioxide and hydrogen are solved, and an industrial by-product is obtained at the same time, so that this process has a relatively low investment cost, and thus it is convenient for industrial application.

### BRIEF DESCRIPTION OF THE DRAWINGS

The drawings, which constitute a portion of the present disclosure, are used to provide a further understanding of the present disclosure. The illustrative embodiments of the present disclosure and the description thereof are used to explain the present disclosure, and do not constitute an improper limitation on the present disclosure.

FIG. 1 shows a schematic diagram of a technological process for capturing and utilizing carbon dioxide according to a first preferred embodiment of the present disclosure.

FIG. 2 shows a schematic diagram of a technological process for capturing and utilizing carbon dioxide according to a second preferred embodiment of the present disclosure, and in this embodiment, a portion of sodium carbonate crystals produced by a system are directly utilized as an industrial by-product.

FIG. 3 shows a schematic diagram of a technological process for capturing and utilizing carbon dioxide according to a fourth preferred embodiment of the present disclosure, and in this embodiment, carbon dioxide in air is concentrated first, and a system is used in combination with an energy storage of compressed air.

FIG. 4 shows a schematic diagram of a technological process for capturing and utilizing carbon dioxide according to Embodiment 1 of the present disclosure.

FIG. 5 shows a schematic flowchart of a method for capturing and utilizing carbon dioxide according to an embodiment of the present disclosure.

FIG. 6 shows a schematic structural diagram of a system for capturing and utilizing carbon dioxide according to an embodiment of the present disclosure.

### DETAILED DESCRIPTION OF THE EMBODIMENTS

It should be noted that, in a case of no conflict, each embodiment in the present disclosure and each feature in each of the embodiment may be combined with each other. The present disclosure will be described in detail below with reference to the embodiments.

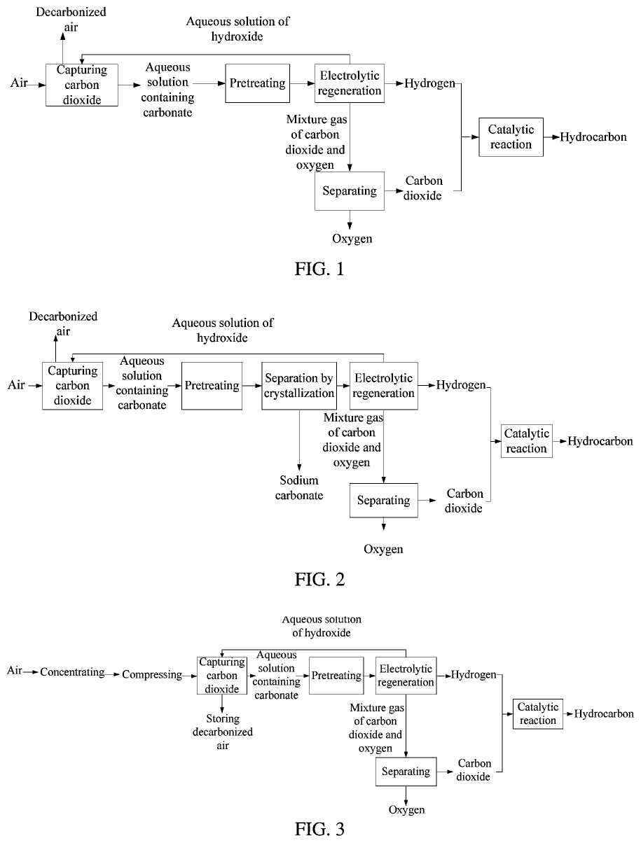

As described in the background, existing methods for capturing carbon dioxide are unable to solve problems of transportation and utilization of the carbon dioxide and an operating cost thereof is high at the same time. In order to solve the foregoing technical problems, as shown in FIG. 1 and FIG. 5, the present disclosure provides a method for capturing and utilizing carbon dioxide which includes: S101, performing, by using an alkaline solution, a capture process on carbon dioxide in a target component, to obtain an aqueous solution containing a carbonate; S103, performing, on the aqueous solution containing the carbonate, an electrolytic regeneration process, to obtain an aqueous solution of hydroxide, carbon dioxide produced by electrolysis, oxygen and hydrogen, and preferably S102, controlling a concentration of the aqueous solution containing the carbonate and an electrolytic voltage in the electrolytic regeneration process to adjust an output ratio of the carbon dioxide produced by the electrolysis and the hydrogen at the same time; and optionally, S104, performing, a catalytic reaction of the carbon dioxide produced by the electrolysis and the hydrogen, to obtain a hydrocarbon, where the hydrocarbon is used as an industrial by-product.

According to the above-mentioned method, the carbon dioxide is capable of being captured from the target component (such as air or a tail gas from burning) to achieve a purpose of reducing emission of the carbon dioxide. Meanwhile, the aqueous solution containing the carbonate is converted to the carbon dioxide, the oxygen, the hydrogen and an aqueous solution of a hydroxide (such as an aqueous solution of sodium hydroxide) by using the electrolytic regeneration process, and the regenerated hydroxide aqueous solution may be returned to the capture process of the carbon dioxide for reuse. Optionally, the carbon dioxide is performed the catalytic reaction with the hydrogen to synthesize the hydrocarbon, so that the problems of the transportation and utilization of the carbon dioxide and the hydrogen are well solved. The generated hydrocarbon may be sold as a by-product, so that an investment cost of a process for capturing and utilizing the carbon dioxide is greatly reduced, thus facilitating industrialization and popularization, and thus an overall production profit is improved. In summary, according to the method for capturing and utilizing the carbon dioxide provided by the present disclosure, not only emission reduction of the carbon dioxide is capable of being realized, but also optionally the problems of the transportation and utilization of the carbon dioxide and hydrogen are solved, and an industrial by-product is obtained at the same time, so that this process has a relatively low investment cost, and thus it is convenient for industrial application and improving the overall production profit.

It should be noted that the hydrogen used in a process of the catalytic reaction may be completely from the electrolytic regeneration process, or may be partially generated by the electrolytic regeneration process, and a remaining portion is transported from outside or completely transported from outside.

In some embodiments, as shown in FIG. 1, the electrolytic regeneration process includes: performing, on the aqueous solution containing the carbonate, the electrolytic regeneration process, to obtain a mixed gas of the carbon dioxide produced by the electrolysis and the oxygen, the hydrogen and the aqueous solution of the hydroxide; and S105 performing, on the mixed gas, a separation process to separate the carbon dioxide produced by the electrolysis and the oxygen. Through the electrolytic regeneration process, the carbon dioxide and the hydrogen may be produced by electrolysis at the same time, which is capable of greatly improving a concentration of the carbon dioxide, makes the carbon dioxide be sustainably produced, and improves a utilization rate of the carbon dioxide.

A reaction principle of the electrolytic regeneration process is as follows: CO₃²⁻+3H₂O→2OH⁻+CO₂↑+O₂↑+2H₂↑. During the electrolytic regeneration process, the oxygen and the carbon dioxide are produced from an anode, and the hydrogen and the sodium hydroxide are produced from a cathode. In order to improve the utilization rate of the carbon dioxide, the oxygen and the carbon dioxide in the mixed gas need to be separated. In some embodiments, a method of the separation process includes but is not limited to one or more of a cryogenic liquefaction method, a catalytic oxidation method and a membrane separation method. Compared with other methods, by adopting the above-mentioned separation method, the investment cost of the overall process may be greatly reduced, and a separation efficiency of the carbon dioxide and the oxygen may be improved.

In some other embodiments, the electrolysis process is a staged electrolysis process. In the staged electrolysis process, the oxygen is generated at the anode of a first-stage electrolytic process, and the carbon dioxide and the residual oxygen is generated at the anode of a second-stage electrolytic process, that is, the staged electrolysis process is two-stages of electrolysis processes which are in series. Through the staged electrolysis process, both the electrolytic regeneration process and the process of separating the carbon dioxide from the mixed gas of the carbon dioxide and the oxygen are capable of being completed in an electrolytic cell, so that a technical process and a process time are greatly shortened, and thus economics of the process is improved.

In order to further improve a yield of the carbon dioxide and the hydrogen in the electrolytic regeneration process, and adjust the output ratio of the carbon dioxide and the hydrogen at the same time, various process parameters may be optimized. In some embodiments, in the electrolytic regeneration process, a voltage of an electrolytic cell ranges from 1.5V to 4V, and preferably, ranges from 2V to 3V, a current density ranges from 1000 A/m² to 10000 A/m², a hydrogen ion concentration (pH) of the aqueous solution containing the carbonate ranges from 7 to 14, and a concentration of the carbonate in the aqueous solution containing the carbonate ranges from 1 mol/L to 10 mol/L. In order to adjust the output ratio and the yield of the carbon dioxide and the hydrogen, the process parameters in the electrolysis process may be adjusted. In some embodiments, the current density ranges from 1500 A/m² to 10000 A/m², preferably ranges from 2000 A/m² to 6000 A/m², and more preferably, ranges from 2000 A/m² to 4000 A/m². In some embodiments, the pH of the aqueous solution containing the carbonate ranges from 7 to 12, and preferably ranges from 7 to 10 or ranges from 8 to 12. Preferably, in the aqueous solution containing the carbonate, the concentration of the carbonate ranges from 1 mol/L to 6.2 mol/L, and more preferably ranges from 1 mol/L to 5 mol/L.

In the electrolytic regeneration process, an electric energy is converted to a required chemical energy to activate a reactivity of the aqueous solution containing the carbonate, so that new carbon dioxide (the carbon dioxide produced by the electrolysis), hydrogen, oxygen and the like are generated. In order to further improve an effect of the electrolytic regeneration process, as shown in FIG. 1, the aqueous solution containing the carbonate may be pretreated (such as concentration adjustment, impurity removal, and preheating). In some embodiments, before performing the electrolytic regeneration process, the method for capturing and utilizing the carbon dioxide further includes: performing, on the aqueous solution containing the carbonate, an impurity removal process. Since the target component for capturing the carbon dioxide may include other impurity components (for example, sulfide, nitride, alkaline earth metal ions such as calcium ions and magnesium ions, and the like) other than the carbon dioxide, the mentioned impurity components may affect the effect of the electrolytic regeneration in the electrolytic regeneration process. Therefore, in order to reduce a risk in this aspect, it is necessary to remove the impurities from the aqueous solution containing the carbonate before the electrolytic regeneration process is performed.

When a content of the alkaline earth metal ions is relatively high, they are easy to precipitate in a form of precipitation during the electrolysis process, and this portion of precipitates will affect the effect of the electrolytic regeneration process and outputs of the carbon dioxide and the hydrogen. In some embodiments, after the impurity removal process, a content of the alkaline earth metal ions in the aqueous solution containing the carbonate is less than or equal to 10 ppm. In some embodiments, the alkaline earth metal ions include, but are not limited to, Ca²⁺ and Mg²⁺.

When a content of the impurity ions is relatively high, they are easy to precipitate in the form of precipitation during the electrolysis process, and this portion of precipitates will affect the effect of the electrolytic regeneration process and the outputs of the carbon dioxide and the hydrogen. In some other embodiments, after the impurity removal process, a content of the impurity ions in the aqueous solution containing the carbonate is less than or equal to 10 ppm, and the impurity ions include alkaline earth metal ions, Al³⁺ and/or Si⁴⁻.

A specific process of impurity removal may be selected according to a composition of the impurities. In some embodiments, a method of the impurity removal includes, but is not limited to a method of filtration, a method of precipitation, or a method of adsorption. Preferably, the precipitation is chemical precipitation.

The electrolytic regeneration process mentioned above-mentioned is performed under a pressure ranges from 1 atm to 40 bar, and is preferably performed under a pressurized condition (in which the pressure ranges from 2 bar to 40 bar). Since carbon dioxide, oxygen and hydrogen will be generated in the electrolytic regeneration process, the process is performed at a higher pressure to facilitate the collection of the above-mentioned gas components on one hand, and on the other hand, it is beneficial to reduce energy consumption of subsequent compression of the gas components.

In some embodiments, between the impurity removal process and the electrolytic regeneration process, the method for capturing and utilizing the carbon dioxide further includes: performing, on a concentration of the aqueous solution containing the carbonate, an adjustment process, where a method of the adjustment process includes diluting with water or concentrating by heating.

In some embodiments, as shown in FIG. 2, the method for capturing and utilizing the carbon dioxide further includes: performing, on a portion of the aqueous solution containing the carbonate, an extraction process, to obtain the carbonate, where the carbonate is directly produced as an industrial by-product. A method for the above-mentioned extraction process includes, but is not limited to, a crystallization method or a recrystallization method and the like. A temperature differential may make the aqueous solution containing the carbonate be supersaturated, thus making the carbonate or bicarbonate crystallize to be separated from the solution. The separated sodium carbonate crystals may be sold as a by-product, for example, as a raw material in a glass industry, so that an economic benefit of the overall process is capable of being further improved, and thus the process cost is reduced.

Since the catalytic reaction of the carbon dioxide and the hydrogen will release a large amount of heat, the aqueous solution containing the carbonate entering the electrolytic cell may also be preheated by this portion of heat. The heat of the catalytic reaction may also be used to generate high-temperature water or steam for utilization of the waste heat. In addition, since an aqueous solution of sodium hydroxide generated in the electrolytic regeneration process also has a certain amount of heat, the aqueous solution of the sodium hydroxide separated from the electrolytic regeneration process may also be used as a heat source for preheating of the aqueous solution containing the carbonate which will enter the electrolytic cell. By adopting the above-mentioned preheating modes, heat energy in the overall process may be more fully utilized, and thus a utilization rate of energy is improved.

In some embodiments, before the electrolytic regeneration process is performed, the method for capturing and utilizing the carbon dioxide further includes: preheating, the aqueous solution containing the carbonate to a temperature ranging from 60° C. to 90° C. The aqueous solution containing the carbonate is preheated to the specific temperature mentioned above, and then the electrolytic regeneration process is performed. Heat generated by electrolysis may be used for generating the high-temperature water for thermal co-generation, so that not only a constant temperature of the electrolytic cell is capable of being maintained, but also a power consumption of the electrolysis is capable of being greatly reduced, and thus a purpose of energy conservation and environmental protection is achieved.

It should be noted that, in a process of pretreatment, the impurity removal process, the concentration adjustment process and the preheating process may be performed in sequence.

In some embodiments, the alkaline solution is an aqueous solution of an alkali metal hydroxide. Compared with other alkaline solutions, the aqueous solution of the alkali metal hydroxide has stronger alkalinity, so that has a better capture effect on the carbon dioxide. The aqueous solution of the alkali metal hydroxide above-mentioned includes, but is not limited to, an aqueous solution of sodium hydroxide and/or an aqueous solution of potassium hydroxide.

Taking the aqueous solution of the sodium hydroxide as an adsorbent for capture for illustrating, a reaction principle of the capture process of the carbon dioxide is as follows: 2NaOH+CO₂=H₂O+Na₂CO₃. An effect of the emission reduction of the carbon dioxide is capable of being realized efficiently and continuously through the above-mentioned capture process. Meanwhile, raw materials used in this process are widely available and low in cost, which is conducive to further reducing the cost for capturing the carbon dioxide. A capture apparatus used in the capture process of the carbon dioxide includes, but is not limited to, a counter-flow cooling tower or a cross-flow cooling tower.

The aqueous solution of the hydroxide generated in the electrolytic regeneration process may also be used as an adsorbent for capture in the capture process, which is capable of realizing cyclic utilization of the adsorbent, so that it is beneficial to greatly reducing the investment cost of the process, and an apparatus involved in the whole process is simple, and thus it is convenient for implementing precise control and industrial application.

In order to further improve the capture effect of the carbon dioxide, in some embodiments, in the capture process of the carbon dioxide, a pH of the alkaline solution ranges from 7 to 14, and preferably is greater than 7 and less than or equal to 10.

In some embodiments, when the electrolytic regeneration process is incomplete, a portion of the carbon dioxide is dissolved in the aqueous solution, so that a product of the electrolytic regeneration process is a bicarbonate (such as NaHCO₃ and KHCO₃). Therefore, when the yield of the carbon dioxide needs to be increased, the electrolytic regeneration process should be made more sufficient as much as possible.

The carbon dioxide, the oxygen and the hydrogen produced in the electrolytic regeneration process may be used in many ways, such as the carbon dioxide may be used in a medical field, a cold storage field and a refrigerant and the like. The hydrogen may be used in the fields of fuel, food, cleaning products and electronic microchip devices. The oxygen may be used in the fields of medical treatment, combustion-supporting and ore mining. In order to make full use of above-mentioned raw materials and produce products with more economic added values at a same time, in some embodiments, the carbon dioxide and the hydrogen are synthesized to a hydrocarbon through a catalytic reaction. In some embodiments, the hydrocarbon includes, but is not limited to, one or more of methane, methanol, gasoline, and aviation fuel.

A temperature and pressure of a process of the catalytic reaction are also different according to a difference of the product final generated. In some embodiments, when the hydrocarbon is methanol, a temperature during the catalytic reaction ranges from 200° C. to 400° C., a pressure ranges from 10 bar to 50 bar, and a molar ratio of the carbon dioxide produced by the electrolysis to the hydrogen ranges from 1:1 to 1:5. A reaction principle of the catalytic reaction which produces the methanol is as follows: CO₂+3H₂→CH₃OH+H₂O. The temperature and pressure of the catalytic reaction and the molar ratio of the carbon dioxide produced by the electrolysis and the hydrogen are limited in the above-mentioned ranges, which is beneficial to further improving a yield of the methanol, thereby the capture cost of the carbon dioxide is further reduced.

Since the catalytic reaction is an exothermic reaction, the reaction heat thereof may be used to generate hot water or steam which are used as industrial by-products. The unreacted carbon dioxide and hydrogen from the catalytic reaction may be recycled back into a catalytic reaction apparatus to increase a production quantity of the final product and conversion rates of the carbon dioxide and the hydrogen. Since an optimal mass ratio of the hydrogen and the carbon dioxide required for generating the methanol is 3:1, the output ratio of different gases produced by the electrolysis (the carbon dioxide and the hydrogen) may be controlled by adjusting a chemical composition of an electrolyte which will enter an inlet for the electrolytic cell and controlling a voltage of the electrolytic cell. The hydrogen and the carbon dioxide with different molar ratios may be used for different downstream catalytic reactions.

In some optional embodiments, as shown in FIG. 3, when the target component is the air, before the capture process is performed, the method for capturing and utilizing the carbon dioxide further includes: performing, on the target component, a concentration process, to obtain a concentrated gas to increase a concentration of the carbon dioxide, and then performing the capture process on the concentrated gas. A concentration of the carbon dioxide in the target component may be improved through the above-mentioned concentration process, so that the capture efficiency of the carbon dioxide in the capture process is greatly improved. Preferably, the concentration process includes: adsorbing, by using an adsorbent, the carbon dioxide in the target component, and then desorbing to obtain the concentrated gas, where a concentration of the carbon dioxide in the concentrated gas ranges from 0.4% to 5% (unit is vol, a volume percentage content).

In some optional embodiments, as shown in FIG. 3, between the concentration process and the capture process, the method for capturing and utilizing the carbon dioxide further includes: performing, on the concentrated gas, a compression process. The compression process is beneficial to improving a solubility of the carbon dioxide in the alkaline solution in the capture process, thereby facilitating further improving the capture rate of the carbon dioxide. Preferably, a pressure of the compression processing ranges from 5 bar to 500 bar. In some embodiments, a compression apparatus used in the above-mentioned compression process is connected to an electrical network, the concentrated gas is compressed by a residual power at a valley load of the electrical network, and is stored in a high-voltage sealing apparatus, and then is released to generate power at a peak power consumption. This is capable of making full use of power resources in the electrical network through a compression energy storage process, and thus the higher economic benefit is obtained.

A tail gas from burning usually includes sulfur-containing oxides, nitrogen-containing oxides and/or dust, these components will affect the subsequent capture process, especially corrode electrodes of an electrolytic cell or may cause catalyst damage for the subsequent catalytic reaction. In order to reduce the influence of the above-mentioned components, in some optional embodiments, when the target component is the tail gas from burning, before the capture process is performed, the method for capturing and utilizing the carbon dioxide further includes: performing, a denitrification treatment and desulfurization treatment, and/or a dust removal treatment on the tail gas from burning.

In some embodiments, the method for capturing and utilizing the carbon dioxide further includes: compressing the carbon dioxide and the hydrogen generated in the electrolytic regeneration process separately, and then performing the catalytic reaction. This is beneficial to improving a reaction rate of the catalytic reaction and a conversion rate of hydrocarbon fuel.

It should be noted that the carbon dioxide produced by the electrolysis refers to a carbon dioxide gas generated in the electrolytic regeneration process.

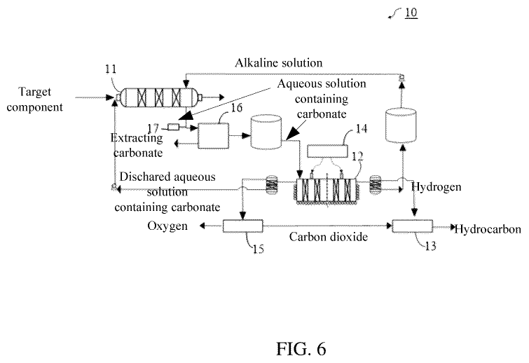

As shown in FIG. 6, another aspect of the present disclosure further provides a system for capturing and utilizing carbon dioxide 10. This system for capturing and utilizing the carbon dioxide includes a carbon dioxide capture apparatus (for example an absorption tower) 11, an electrolytic regeneration unit 12, and optionally a catalytic apparatus (for example a synthesis reactor) 13. The carbon dioxide capture apparatus 11 is provided with an inlet for an alkaline solution, an inlet for a target component and a discharge port for an aqueous solution containing a carbonate. The electrolytic regeneration unit 12 is provided with a first inlet for the aqueous solution containing the carbonate, an outlet for carbon dioxide produced by electrolysis, an outlet for oxygen, an outlet for hydrogen and a discharge port for an aqueous solution of hydroxide, and the first inlet for the aqueous solution containing the carbonate is in communication with the discharge port for the aqueous solution containing the carbonate through a transport pipeline of the aqueous solution containing the carbonate. In some embodiments, the system above-mentioned further includes a voltage regulating apparatus 14. The voltage regulating apparatus (for example a voltage regulator) 14 is configured to regulate a voltage in an electrolytic regeneration process to adjust an output ratio of the carbon dioxide produced by electrolysis and the hydrogen. The catalytic apparatus 13 is provided with a catalytic inlet and an outlet for the hydrocarbon, and the catalytic inlet is respectively in communication with the outlet for the carbon dioxide produced by the electrolysis and the outlet for the hydrogen.

According to the system for capturing and utilizing the carbon dioxide 10 above-mentioned, the carbon dioxide is capable of being captured from the target component (such as air or a tail gas from burning) to achieve a purpose of reducing emission of the carbon dioxide. Meanwhile, the electrolytic regeneration unit 12 is configured to convert the aqueous solution containing the carbonate to carbon dioxide, oxygen, hydrogen, and an aqueous solution of hydroxide (such as an aqueous solution of sodium hydroxide), and the generated aqueous solution of the hydroxide may be returned to a capture process of the carbon dioxide for reuse. Optionally, the carbon dioxide and the hydrogen are performed a catalytic reaction in the catalytic apparatus 13 to synthesize a hydrocarbon, which is capable to well solve the problems of the transportation and utilization of the carbon dioxide and the hydrogen. The generated hydrocarbon may be sold as a by-product, so that the investment cost of the process of the capture and utilization of the carbon dioxide may be greatly reduced, thus facilitating industrialization and popularization, and thus the overall production profit is improved. In summary, the system for capturing and utilizing the carbon dioxide 10 provided by the present disclosure is not only capable of realizing the emission reduction of the carbon dioxide, optionally is capable of solving the problems of the transportation and utilization of the carbon dioxide and the hydrogen, and is also capable of obtaining an industrial by-product, such as a direct output of sodium carbonate or an output of the hydrocarbon, so that this process has a relatively low investment cost, which is convenient for an industrial application, and thus the overall production profit is improved.

In some embodiments, when a voltage for electrolysis is regulated by the voltage regulating apparatus 14 to make the electrolytic regeneration apparatus be able to perform a staged electrolysis process, the carbon dioxide and the oxygen is capable of being produced in stages. At this time, it is not necessary to provide a separation apparatus 15. The electrolytic regeneration unit 12 only includes an electrolytic regeneration apparatus (for example an electrolytic cell), and the outlet for the carbon dioxide produced by the electrolysis, the outlet for the oxygen, the outlet for the hydrogen and discharge port for the aqueous solution of the hydroxide are all provided on the electrolytic regeneration apparatus.

In some embodiments, when the electrolytic regeneration apparatus cannot perform a staged electrolysis process, the electrolytic regeneration unit 12 includes an electrolytic regeneration apparatus and a separation apparatus 15 (for example a separator). The electrolytic regeneration apparatus is provided with the first inlet for the aqueous solution containing the carbonate, the outlet for the hydrogen, a discharge port for anode gases and the discharge port for the aqueous solution of the hydroxide. The anode gases include the carbon dioxide produced by electrolysis and the oxygen. The separation apparatus 15 is provided with an inlet for a gas to be separated, an outlet for the carbon dioxide produced by the electrolysis and an outlet the oxygen, and the inlet for the gas to be separated is in communication with the discharge port for the anode gases. The carbon dioxide and the hydrogen may be simultaneously generated by the electrolytic regeneration apparatus, which is capable of greatly improving a concentration of the carbon dioxide, and enabling the carbon dioxide be sustainably produced, and thus the utilization rate of the carbon dioxide is improved. In some embodiments, the separation apparatus 15 includes, but is not limited to, one or more of a cryogenic apparatus, a catalytic oxidation apparatus, an adsorption apparatus and a membrane separation apparatus. Compared with other separation apparatus, the separation apparatus 15 mentioned above may greatly reduce the investment cost of the overall process and improve a separation efficiency of the carbon dioxide and the oxygen.

Since the target component for carbon dioxide capture may include other impurity components (such as sulfide, nitride and alkaline earth metal ions for example calcium ions and magnesium ions, and the like) besides the carbon dioxide. The above-mentioned impurity components may affect an effect of electrolytic regeneration in the electrolytic regeneration process. In order to reduce a risk of this aspect, in some embodiments, the system for capturing and utilizing the carbon dioxide further includes an impurity removal apparatus. The impurity removal apparatus is disposed on the transport pipeline of the aqueous solution containing the carbonate. A specific process for impurity removal may be selected according to a composition of the impurities. In some embodiments, the impurity removal apparatus includes, but is not limited to, a filtration apparatus, a precipitation apparatus or an adsorption apparatus.

Since the electrolytic regeneration process and the catalytic reaction process will release a large amount of heat, in order to further improve a utilization rate of the heat of the overall process, in some embodiments, the system for capturing and utilizing the carbon dioxide 10 further includes a first heat exchange apparatus (for example a heat exchanger). The first heat exchange apparatus is disposed on the transport pipeline of the aqueous solution containing the carbonate. The heat in the aqueous solution containing the carbonate is recycled by the first heat exchange apparatus, so that a utilization rate of energy is improved. In some embodiments, a hot medium inlet for the first heat exchange apparatus is connected to the discharge port for the aqueous solution of the hydroxide, to make the aqueous solution of the hydroxide discharged from the discharge port for the aqueous solution of the hydroxide exchange heat with the aqueous solution containing the carbonate in the transport pipeline of the aqueous solution containing the carbonate. In some other embodiments, the system for capturing and utilizing the carbon dioxide 10 includes a catalytic apparatus 13 and a second heat exchange apparatus (for example a heat exchanger). The second heat exchange apparatus is disposed on the transport pipeline of the aqueous solution containing the carbonate. In some embodiments, a hot medium inlet for the second heat exchange apparatus is connected to the outlet for the hydrocarbon, to make the hydrocarbon discharged from the outlet for the hydrocarbon exchange heat with the aqueous solution containing the carbonate in the transport pipeline of the aqueous solution containing the carbonate.

In the electrolytic regeneration process, a concentration and a pH of the aqueous solution containing the carbonate and an electrolysis voltage will all affect the effect of the electrolytic regeneration process. In order to improve yields of the carbon dioxide and the hydrogen in the electrolytic regeneration process and adjust a ratio of the two, the concentration and the pH of the aqueous solution containing the carbonate and the electrolysis voltage need to be adjusted as required. In some embodiments, the system for capturing and utilizing the carbon dioxide 10 further includes a concentration adjusting apparatus 17 of the aqueous solution containing the carbonate and a pressure regulating apparatus (also called a voltage regulator). The concentration adjusting apparatus of the aqueous solution containing the carbonate is disposed on the transport pipeline of the aqueous solution containing the carbonate, and is configured to adjusting a concentration and a pH of the aqueous solution containing the carbonate. The above-mentioned pressure regulating apparatus is configured to regulate the electrolysis voltage in the electrolytic apparatus. In some embodiments, the concentration adjusting apparatus 17 of the aqueous solution containing the carbonate is a dilution apparatus (for example a diluting tank) or a concentration apparatus (for example a negative pressure evaporator).

In some embodiments, the system for capturing and utilizing the carbon dioxide 10 further includes a carbonate extraction apparatus. The carbonate extraction apparatus is provided with a second inlet for the aqueous solution containing the carbonate, and the second inlet for the aqueous solution containing the carbonate is in communication with the discharge port for the aqueous solution containing the carbonate. By providing the carbonate extraction apparatus, excess carbonate may be separated out and directly produced as an industrial by-product, thereby facilitating further reducing the capture cost of the carbon dioxide. The extraction apparatus above mentioned includes, but is not limited to, a crystallization apparatus (for example a carbonate evaporative crystallizer) or a recrystallization apparatus (for example a carbonate solvent crystallization apparatus). The carbonate may be crystallized in the crystallization apparatus or the recrystallization apparatus through a temperature difference, to make the carbonate be precipitated and/or further purified. In addition, since in the aqueous solution containing the carbonate discharged from the capture apparatus includes a portion of the aqueous solution of the hydroxide, it may also be returned to an absorption tower as an adsorbent for capture.

Since products of the electrolytic regeneration process includes an aqueous solution of a hydroxide, this raw material may also be used in the capture process of the carbon dioxide, so that a utilization rate of the aqueous solution of the hydroxide is further improved, and thus a utilization rate of the raw materials and a recyclability of the capture process of the carbon dioxide are improved at the same time. In some embodiments, the discharge port for the aqueous solution of the hydroxide is in communication with the inlet for the alkaline solution.

In some embodiments, when the target component is the air, the system for capturing and utilizing the carbon dioxide 10 further includes a concentration unit. The concentration unit is provided with an inlet for a gas to be concentrated and an outlet for a concentrated gas, the outlet for the concentrated gas is in communication with the inlet for the target component through a transport pipeline of the concentrated gas, and the concentration unit is configured to improve a content of the carbon dioxide in the target component. The concentration of the carbon dioxide in the target component may be improved through the concentration unit, so that the capture efficiency of the carbon dioxide in the capture process is greatly improved. In order to further improve a concentration efficiency of the carbon dioxide, in some embodiments, the concentration unit includes a carbon dioxide adsorption apparatus and a desorption apparatus. The carbon dioxide adsorption apparatus is provided with the inlet for the gas to be concentrated, and is configured to adsorb the carbon dioxide in the target component. The desorption apparatus is disposed downstream of the carbon dioxide adsorption apparatus and provided with an outlet for the concentrated gas, which is configured to desorb the carbon dioxide being adsorbed in the carbon dioxide adsorption apparatus.

In some embodiments, the system for capturing and utilizing the carbon dioxide 10 further includes a first compression apparatus (for example a compressor) disposed on the transport pipeline of the concentrated gas. Through the first compression apparatus, a solubility of the carbon dioxide in the alkaline solution during the capture process is improved, so that the capture rate of the carbon dioxide is further improved. In some embodiments, the first compression apparatus above-mentioned is connected to an electrical network, the concentrated gas is compressed by a residual power at a valley load of the electrical network, and is stored in a high-voltage sealing apparatus, and then is released to generate power at a peak power consumption. This is capable of making full use of power resources in the electrical network through a compression energy storage process, and thus the higher economic benefit is obtained.

In some embodiments, when the target component is the tail gas from burning, the system for capturing and utilizing the carbon dioxide 10 further includes a dust removal apparatus (for example a fabric filter, an electrostatic dust collector, a cartridge dust collector, a wet electrostatic precipitator), a desulfurization apparatus (for example a fiberglass desulfurization tower, a limestone-gypsum desulfurization apparatus, a wet desulfurization device), a denitrification apparatus (for example a denitration and dust removal device, a polymer denitration machine, a selective catalytic reduction (SCR) system, a selective nocatalytic reduction (SCNR) system) and a transport pipeline of the target component in communication with inlet for the target component. The dust removal apparatus, the desulfurization apparatus and the denitrification apparatus are disposed on transport pipeline of the target component. It should be noted that the dust removal apparatus, the desulfurization apparatus and the denitrification apparatus above-mentioned are all disposed on transport pipeline of the target component, and the sequence of the three apparatuses may be sorted according to requirements.

In some embodiments, the system for capturing and utilizing the carbon dioxide 10 further includes a collection apparatus. The collection apparatus is provided with a collection port, and the collection port is in communication with the oxygen outlet for collecting oxygen. The oxygen is collected by the collection apparatus for subsequent utilization, so that the economic value of the overall process is capable of being further improved.

In some embodiments, the system for capturing and utilizing the carbon dioxide 10 further includes a carbon dioxide compression apparatus (for example a compressor) and a hydrogen compression apparatus (for example a compressor). The carbon dioxide compression apparatus is provided with an inlet for the carbon dioxide produced by the electrolysis and an outlet for compressed carbon dioxide, the inlet for the carbon dioxide produced by the electrolysis is in communication with the outlet for the carbon dioxide produced by the electrolysis of the electrolytic regeneration unit 12, and the outlet for the compressed carbon dioxide is in communication with the catalytic inlet. The hydrogen compression apparatus is provided with an inlet for hydrogen and an outlet for compressed hydrogen, the inlet for the hydrogen is in communication with the outlet for the hydrogen of the electrolytic regeneration unit 12, and the outlet for the compressed hydrogen is in communication with the catalytic inlet. The carbon dioxide and the hydrogen after being compressed is transported to the catalytic apparatus 13, which is conducive to further improving the reaction rate of the catalytic reaction and a conversion rate of hydrocarbon fuel.

A preferred process of capture and utilization of the carbon dioxide provided in the present disclosure is shown in FIG. 1 (the target component is the air). Adopting the above-mentioned method, a system for producing carbon-negative fuel oil may be constructed, which may capture 100,000 tons of the carbon dioxide from the air per year, it is equivalent to a total amount of the carbon dioxide absorbed by 5 million adult trees per year. This process may reduce a content of the carbon dioxide in the atmosphere, which may be used to capture the carbon dioxide generated in industries such as power stations, chemical stations, cement, steel and the like, and at the same time produce the hydrogen and synthesize a negative carbon energy, thus solving a problem of the carbon emission in transportation industries such as aviation, automobiles, ships and the like. Application scenarios of this process include but are not limited to: (1) Using a renewable energy or nuclear energy to capture carbon dioxide from the air to produce the negative carbon energy source, to reduce the emission of the carbon dioxide of aircrafts, ships, automobiles and other transportations. (2) Using this apparatus to capture carbon dioxide from the power station or an industrial system and convert the carbon dioxide to zero-carbon fuel to solve the problems of capture, transportation and utilization of industrial carbon dioxide. (3) Since the carbon dioxide and the hydrogen is simultaneously generated, the present disclosure may also solve problems of storage and transportation and utilization of hydrogen produced by electrolyzing water. (4) This technique may be used to solve a problem of long-term storage and transportation of a renewable energy.

### Embodiment 1

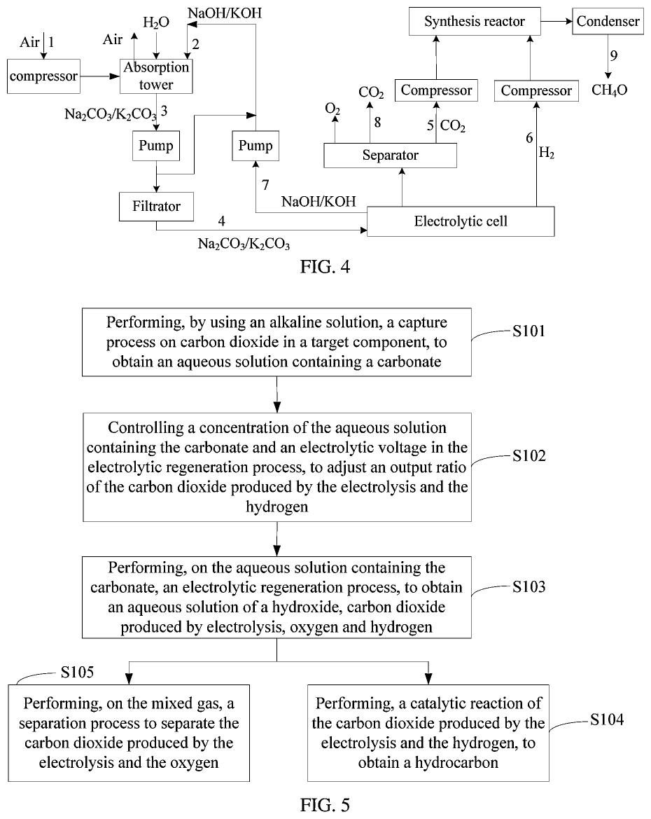

As shown in FIG. 4, a schematic diagram of a technological process for capturing and utilizing carbon dioxide is provided, and a temperature, pressure, flow, and composition of each process in the technological process are shown in Table 1.

### Capture Process:

After the air is compressed by a compressor, compressed air is obtained and transported to an absorption tower. In the absorption tower, the compressed air is reacted with an alkaline solution (an aqueous solution of sodium hydroxide/potassium hydroxide) to convert to an aqueous solution containing a carbonate (an aqueous solution of sodium carbonate or potassium carbonate). A portion of the aqueous solution containing the carbonate is pump into a filtration apparatus (for example a resin tower or an ultrafiltration membrane) for filtering, and then is transported to an electrolytic cell after being filtered, and another portion of the aqueous solution containing the carbonate is returned to the absorption tower again as a circulating absorbent for capture.

### Electrolytic Regeneration Process:

The aqueous solution containing the carbonate (such as the aqueous solution of sodium carbonate or potassium carbonate) is performed electrolytic regeneration in the electrolytic cell to produce a mixed gas of carbon dioxide and oxygen at an anode and hydrogen at a cathode. The mixed gas of the carbon dioxide and the oxygen is separated in a separator (such as separated by a cryogenic apparatus), then the carbon dioxide and a mixed gas of the oxygen and a small amount of carbon dioxide is obtained. A portion of the aqueous solution of the sodium hydroxide or the aqueous solution of the potassium hydroxide generated by the electrolytic regeneration process is pumped into the absorption tower as an absorbent for capture.

### Synthesis of Hydrocarbons:

The separated purer carbon dioxide is transported to a compressor for compression, the hydrogen generated in the electrolytic regeneration process is compressed, and then the two is performed a catalytic reaction (a catalyst for catalytic hydrogenation is ZnZrO/ZSM⁻⁵, a pressure ranges from 25 bar to 35 bar, and a temperature ranges from 200° C. to 300° C.) in a synthesis reactor to obtain a product gas. After the product gas is cooled in a condenser, methanol is obtained.

TABLE 1
Parameters of material streams of a System

| Material | | | | Molar component (N₂, O₂, CO₂, |
|---|---|---|---|---|
| streams | Temperature/° C. | Pressure/MPa | Flow kg/h | H₂O, H₂, NaOH, Na₂CO₃, CH₄O) |
| 1 | 15 | 0.1 | 941.6 | 0.775, 0.208, 0.001, 0.007, 0, 0, 0, 0 |
| 2 | 60 | 0.15 | 201.7 | 0, 0, 0, 0.942, 0, 0.021, 0.036, 0 |
| 3 | 40 | 0.1 | 188.5 | 0, 0, 0, 0.938, 0, 0.021, 0.04, 0 |
| 4 | 40 | 0.15 | 3.77 | 0, 0, 0, 0.938, 0, 0.021, 0.04, 0 |
| 5 | 80 | 0.1 | 0.102 | 0, 0, 1, 0, 0, 0, 0, 0 |
| 6 | 80 | 0.1 | 0.014 | 0, 0, 0, 0, 1, 0, 0, 0 |
| 7 | 80 | 0.1 | 3.34 | 0, 0, 0, 0.893, 0, 0.107, 0, 0 |
| 8 | 80 | 0.1 | 0.204 | 0, 0, 1, 0, 0, 0, 0, 0 |
| 9 | 15 | 3 | 0.115 | 0, 0, 0, 0.5, 0, 0, 0, 0.5 |

Performance parameters of System

| Item | Value |
|---|---|
| capture amount of CO₂ kg/y | 2680 |
| Production quantity of Methanol kg/y | 1007 |
| Air flow of absorption tower/kg/h | 941.6 |
| Power consumption of absorption tower/kWh/y | 217 |
| Power of electrolytic cell/kW | 0.83 |
| Power consumption of electrolytic cell/kWh/y | 7236 |

### Embodiment 2

Differences from Embodiment 1 are: in the electrolytic regeneration process, the voltage of the electrolytic cell is 2 V, the pH of the aqueous solution containing the carbonate is 7, the current density is 4000 A/m², and the concentration of the carbonate in the aqueous solution containing the carbonate is 5 mol/L.

### Embodiment 3

Differences from Example 1 is: in the electrolytic regeneration process, the voltage of the electrolytic cell is 1.5 V, the pH of the aqueous solution containing the carbonate is 8, the current density is 6000 A/m², and the concentration of the carbonate in the aqueous solution containing the carbonate is 6.2 mol/L.

### Embodiment 4

Differences from Example 1 is: in the electrolytic regeneration process, the voltage of the electrolytic cell is 4 V, the pH of the aqueous solution containing the carbonate is 10, the current density is 6000 A/m², and the concentration of the carbonate in the aqueous solution containing the carbonate is 6.2 mol/L.

### Embodiment 5

Differences from Example 1 is: in the electrolytic regeneration process, the voltage of the electrolytic cell is 2 V, the pH of the aqueous solution containing the carbonate is 12, the current density is 4000 A/m², and the concentration of the carbonate in the aqueous solution containing the carbonate is 5 mol/L.

### Embodiment 6

Differences from Example 1 is: in the electrolytic regeneration process, the voltage of the electrolytic cell is 3 V, the pH of the aqueous solution containing the carbonate is 14, the current density is 2000 A/m², and the concentration of the carbonate in the aqueous solution containing the carbonate is 1 mol/L.

The technological processes of capture and utilization of the carbon dioxide in Embodiments 2 to 6 provided in the present disclosure are same as Embodiment 1, and performance parameters of the system of Embodiments 2 to 6 are shown in Table 2.

TABLE 2

| | | | | Power |
|---|---|---|---|---|
| | Capture | Production | Powe of | consumption |
| | amount | quantity of | Electrolytic | of |
| | of | Methanol | cell | Electrolytic |
| | CO₂ kg/y | kg/y | r/kW | cell/kWh/y |
| Embodiment 2 | 2466 | 926 | 0.76 | 6652 |
| Embodiment 3 | 3216 | 1208 | 1.02 | 8851 |
| Embodiment 4 | 3752 | 1410 | 1.20 | 10927 |
| Embodiment 5 | 3323 | 1249 | 1.08 | 9415 |
| Embodiment 6 | 4020 | 1511 | 1.37 | 11830 |

From the foregoing description, it may be seen that the foregoing embodiments of the present disclosure achieve following technical effects:
(1) The carbon dioxide in the air and the tail gas from burning is absorbed by using the alkaline solution to generate the aqueous solution containing the carbonate, and by electrolysis of the aqueous solution containing the carbonate, the carbon dioxide is generated, the alkaline solution adsorbent is regenerated, and the hydrogen is generated at the same time.
(2) The apparatus or system may adjust the chemical composition of the electrolyte flowing into the inlet for the electrolytic cell and control the voltage of the electrolytic cell to control the output ratio of different electrolytic gases (the carbon dioxide and the hydrogen). The hydrogen and the carbon dioxide with different molar ratios may be used for different downstream catalytic reactions. The CO₂ and the O₂ generated by the alkaline electrolytic cell needs to be performed gas separation, and the CO₂ and the H₂ are synthesized to generate hydrocarbon fuel such as methane, methanol, gasoline, aviation fuel and the like.
(3) The synthesis catalytic reaction of the CO₂ and the H₂ is the exothermic reaction, and the heat thereof may be reasonably recycled to generate hot water or steam, or be used for preheating the electrolytic cell of the aqueous solution containing the carbonate, to make the electrolytic cell of the aqueous solution containing the carbonate operate at an optimal temperature to achieve a highest efficiency.
(4) This technology for capturing and utilizing the carbon dioxide simultaneously implements the capture and the utilization of the carbon dioxide, and the carbon dioxide captured from the air or fume may be directly produced as an industrial by-product in a form of solid soda ash, or be directly output in a form of liquid hydrocarbon fuel, thereby production profit of the whole apparatus or system is improved and a diversity of outputs of the whole apparatus or system is realized. Meanwhile, the problems of the transportation and utilization of the carbon dioxide are solved, and the problems of the transportation and utilization of the hydrogen in a field of hydrogen production by electrolyzing water are also solved.

It should be noted that the terms “first”, “second”, and the like in the specification and claims of the present disclosure are used to distinguish similar objects, and are not necessarily used to describe a specific order or sequence. It should be understood that the terms used in this way are interchangeable under appropriate circumstances, so that the embodiments of the present disclosure described herein can be implemented in an order other than those described herein.

The foregoing are only preferred embodiments of the present disclosure and are not intended to limit the present disclosure, and for a person skilled in the art, the present disclosure may have various modifications and changes. Any modification, equivalent replacement or improvement made within the spirit and principle of the present disclosure shall fall within the protection scope of the present disclosure.

What is claimed is:

1. A method for capturing and utilizing carbon dioxide, comprising:
performing, by using an alkaline solution, a capture process on carbon dioxide in a target component, to obtain an aqueous solution containing a carbonate;
performing, on the aqueous solution containing the carbonate, an electrolytic regeneration process, to obtain an aqueous solution of a hydroxide, carbon dioxide produced by electrolysis, oxygen and hydrogen; and
performing a catalytic reaction of the carbon dioxide produced by the electrolysis and the hydrogen to obtain a hydrocarbon.

2. The method for capturing and utilizing the carbon dioxide according to claim 1, further comprising: controlling a concentration of the aqueous solution containing the carbonate and an electrolytic voltage in the electrolytic regeneration process, to adjust an output ratio of the carbon dioxide produced by the electrolysis and the hydrogen.

3. The method for capturing and utilizing the carbon dioxide according to claim 1, wherein the electrolytic regeneration process comprises:
performing, on the aqueous solution containing the carbonate, the electrolytic regeneration process, to obtain a mixed gas of the carbon dioxide produced by the electrolysis and the oxygen, the hydrogen and the aqueous solution of the hydroxide; and
performing, on the mixed gas, a separation process, to separate the carbon dioxide produced by the electrolysis and the oxygen in the mixed gas.

4. The method for capturing and utilizing the carbon dioxide according to claim 1, wherein the electrolytic regeneration process is a staged electrolysis process.

5. The method for capturing and utilizing the carbon dioxide according to claim 1, wherein in the electrolytic regeneration process, a voltage of an electrolytic cell ranges from 1.5V to 4V, a current density ranges from 1000 A/m² to 10000 A/m², a pH of the aqueous solution containing the carbonate ranges from 7 to 14, and a concentration of the carbonate in the aqueous solution containing the carbonate ranges from 1 mol/L to 10 mol/L.

6. The method for capturing and utilizing the carbon dioxide according to claim 1, wherein before performing the electrolytic regeneration process, the method for capturing and utilizing the carbon dioxide further comprises: performing, on the aqueous solution containing the carbonate, an impurity removal process, wherein after the impurity removal process, a content of alkaline earth metal ions in the aqueous solution containing the carbonate is less than or equal to 10 ppm.

7. The method for capturing and utilizing the carbon dioxide according to claim 1, wherein the electrolytic regeneration process is performed under a pressure ranges from 1 atm to 40 bar.

8. The method for capturing and utilizing the carbon dioxide according to claim 1, further comprising: performing, on a portion of the aqueous solution containing the carbonate, an extraction process, to obtain the carbonate.

9. The method for capturing and utilizing the carbon dioxide according to claim 1, wherein a pH of the alkaline solution ranges from 7 to 14.

10. The method for capturing and utilizing the carbon dioxide according to claim 1, wherein the hydrocarbon is methanol, a temperature in the catalytic reaction ranges from 200° C. to 400° C., a pressure ranges from 10 bar to 50 bar, and a molar ratio of the carbon dioxide produced by the electrolysis to the hydrogen ranges from 1:1 to 1:5.

11. A system for capturing and utilizing carbon dioxide, comprising:
a carbon dioxide capture apparatus, wherein the carbon dioxide capture apparatus is provided with an inlet for an alkaline solution, an inlet for a target component and a discharge port for an aqueous solution containing a carbonate;
an electrolytic regeneration unit, wherein the electrolytic regeneration unit is provided with a first inlet for the aqueous solution containing the carbonate, an outlet for carbon dioxide produced by electrolysis, an outlet for oxygen, an outlet for hydrogen and a discharge port for an aqueous solution of a hydroxide, and the first inlet for the aqueous solution containing the carbonate is in communication with the discharge port for the aqueous solution containing the carbonate through a transport pipeline of the aqueous solution containing the carbonate; and
a catalytic apparatus, wherein the catalytic apparatus is provided with a catalytic inlet and an outlet for the hydrocarbon, and the catalytic inlet is respectively in communication with the outlet for the carbon dioxide produced by the electrolysis and the outlet for the hydrogen.

12. The system for capturing and utilizing the carbon dioxide according to claim 11, further comprising:
a voltage regulating apparatus, wherein the voltage regulating apparatus is configured to regulate a voltage in an electrolytic regeneration process to adjust an output ratio of the carbon dioxide produced by electrolysis and the hydrogen.

13. The system for capturing and utilizing the carbon dioxide according to claim 11, wherein the voltage regulating apparatus is capable of making the electrolytic regeneration unit perform a staged electrolysis process, the electrolytic regeneration unit is a first electrolytic regeneration apparatus, and the first electrolytic regeneration apparatus is provided with the first inlet for the aqueous solution containing the carbonate, the outlet for the hydrogen, the outlet for the carbon dioxide produced by the electrolysis, the outlet for the oxygen and the discharge port for the aqueous solution of the hydroxide.

14. The system for capturing and utilizing the carbon dioxide according to claim 11, wherein the electrolytic regeneration unit does not perform a staged electrolysis process, and the electrolytic regeneration unit comprises:
a second electrolytic regeneration apparatus, wherein the second electrolytic regeneration apparatus is provided with the first inlet for the aqueous solution containing the carbonate, the outlet for the hydrogen, a discharge port for anode gases and the discharge port for the aqueous solution for the hydroxide, wherein the anode gases comprise the carbon dioxide produced by electrolysis and the oxygen; and
a separation apparatus, wherein the separation apparatus is provided with an inlet for a gas to be separated, an outlet for the carbon dioxide produced by the electrolysis and an outlet the oxygen, and the inlet for the gas to be separated is in communication with the discharge port for the anode gas.

15. The system for capturing and utilizing the carbon dioxide according to claim 11, further comprising an impurity removal apparatus, wherein the impurity removal apparatus is disposed on the transport pipeline of the aqueous solution containing the carbonate.

16. The system for capturing and utilizing the carbon dioxide according to claim 11, further comprising a first heat exchange apparatus, wherein the first heat exchange apparatus is disposed on the transport pipeline of the aqueous solution containing the carbonate, and a hot medium inlet for the first heat exchange apparatus is connected to the discharge port for the aqueous solution of the hydroxide, to make the aqueous solution of the hydroxide discharged from the discharge port for the aqueous solution of the hydroxide exchange heat with the aqueous solution containing the carbonate in the transport pipeline of the aqueous solution containing the carbonate.

17. The system for capturing and utilizing the carbon dioxide according to claim 11, further comprising the catalytic apparatus and a second heat exchange apparatus, wherein the second heat exchange apparatus is disposed on the transport pipeline of the aqueous solution containing the carbonate, and a hot medium inlet for the second heat exchange apparatus is connected to the outlet for the hydrocarbon, to make the hydrocarbon discharged from the outlet for the hydrocarbon exchange heat with the aqueous solution containing the carbonate in the transport pipeline of the aqueous solution containing the carbonate.

18. The system for capturing and utilizing the carbon dioxide according to claim 11, further comprising:
a concentration adjusting apparatus of the aqueous solution containing the carbonate disposed on the transport pipeline of the aqueous solution containing the carbonate, wherein the concentration adjusting apparatus of the aqueous solution containing the carbonate is configured to adjust a concentration and a pH of the aqueous solution containing the carbonate.

19. The system for capturing and utilizing the carbon dioxide according to claim 11, further comprising a carbonate extraction apparatus, wherein the carbonate extraction apparatus is provided with a second inlet for the aqueous solution containing the carbonate, and the second inlet for the aqueous solution containing the carbonate is in communication with the discharge port for the aqueous solution containing the carbonate.

20. The system for capturing and utilizing the carbon dioxide according to claim 11, wherein the discharge port for the aqueous solution of the hydroxide is in communication with the inlet for the alkaline solution.
